(19) United States
(12) Patent Application Publication
(10) Pub. No.: US 2008/0213416 A1
(43) Pub. Date: Sep. 4, 2008

(54) Composition Containing an Extract of Rubi Fructus for Preventing and Treating Anxiety, Depression and Dementia, and Improving Memory

(76) Inventor: Sung-Jin Kim, Seoul (KR)

Correspondence Address: STETINA BRUNDA GARRED & BRUCKER, 75 ENTERPRISE, SUITE 250, ALISO VIEJO, CA, 92656, US

(21) Appl. No.: 12/120,449

(22) Filed: May 14, 2008

Related U.S. Application Data
(62) Division of application No. 11/648,325, filed on Dec. 29, 2006.

(30) Foreign Application Priority Data
May 8, 2006 (KR) 10-2006-41105

(51) Int. Cl. A61K 36/00; A61P 25/00

(52) U.S. Cl. 424/777

(57) ABSTRACT

Disclosed is a composition containing an extract of Rubi Fructus for preventing and treating anxiety, depression and dementia and improving memory. The composition can be used as drugs and dietary supplements which induce prophylactic and therapeutic effects on anxiety, depression and dementia as well as memory-improving effect in the moderns afflicted with the modification of neurotransmitter releases and brain damage caused by external environmental factors including various kinds of stresses, menopause, drinking alcohols, smoking cigarettes and others.

### CROSS-REFERENCE TO RELATED APPLICATIONS

This application is based on and claims priority to Korean Patent Application No. 10-2006-41105 filed on May 8, 2006 in the Korean Intellectual Property Office (KIPO), the entire contents of which are hereby incorporated by reference.

### STATEMENT RE: FEDERALLY SPONSORED RESEARCH/DEVELOPMENT

Not Applicable

### BACKGROUND

1. Field of the Invention

The present invention relates to a composition containing an extract of Rubi Fructus for preventing and treating anxiety, depression and dementia and improving memory, more particularly to a composition that can contains an extract of Rubi Fructus inducing prophylactic and therapeutic effects on anxiety, depression and dementia as well as memory-improving effect and can be thus used for preventing and treating anxiety, depression and dementia and improving memory.

2. Description of the Related Art

From ancient to modern times, melancholia has been recognized as showing both anxiety and depression symptoms (Glass G A. 1994. A conceptual of anxiety and depression. In; deb Boer J A, Sitsen J M A. Handbook of depression and anxiety. New York, N.Y.: Marcel Dekker. P 1-44). There are often comorbidities between anxiety and depression. 10-25% of the total population of the world will experience some sort of depression once in the life, and 5-6% thereof may be suffering from depression now. Depression may afflict people of all ages and both sexes. Generally, depression occurs in people in their age 30s and 40s, however, it tends to occur early in women. Depression generally lasts more than 6 months, and is accompanied by psychological distress and physical impairment. Therefore, if people are not treated appropriately for depression, it may lead them to commit suicide. Anxiety shows symptoms expressed emotionally and physically and through thoughts. Emotional symptoms include instability, anger, irritability and others, and physical symptoms include irregular and rapid beating of heart, digestive disorders, diarrhea, constipation, excessive hand sweating, tremors, cold extremities, muscle tension, headache, rear neck or shoulder pains, chest pressure or pains, xerostomia, shortness of breath, dizziness, insomnia and others.

It is known that anxiety and depression are closely related with memory impairments. There are provided evidences that depressive or anxiety complaints may occur due to memory decline [Schmand, B., Jonker, C., Geerlinhs, M. I., Lindeboom, J. Subjective memory complaints in the elderly, depressive symptoms and future dementia. Br. J. Psychiatry 171, 373-376, 1997; Harwood, D. G., Braker, W. W., Ownby, R. L., Duara, R. Relationship of behavioral and psychological symptoms to cognitive impairment and functional status in Alzheimer's disease. Int. J. Geriatr. Psychiatry 15, 393-400, 2000; Clarnette, R. M., Almeida, O. P., Forstl, H., Paton, A., Martins, R. N. Clinical characteristics of individuals with subjective memory decline in Western Australia: results from a cross-sectional study. Int. J. Geriatr. Psychiatry 16, 168-174, 2001].

Further, it is reported that anxiety and depression are induced as a response for the onset of memory decline, rather than they are causes of memory decline [Devanand, D. P., Sano, M., tang, M. X. et al. Depressed mood and the incidence of Alzheimer's disease: what is consensual? What is controversial? What is practical? J. Clin. Psychiatry 59, 6-18, 1996; Jorm, A. F., Christensen, H., Korten, A. E., Hendersen, A. S., Jacomb, P. A., Mackinnon, A. Do cognitive complaints either predict future cognitive decline or reflect past cognitive decline? A longitudinal study of an early community sample. Psychol. Med. 27, 91-98, 1997]. The interaction between memory impairments and anxiety is known to be bi-directional, because anxiety brings the loss of memory [Derousene, C., Lacomblez, L., Thibault, S., LePonncin, M. Memory complaints in young and elderly subjects. Int. J. geriatr. Psychiatry, 14, 291-3-1,1999] and the loss of memory also induces anxiety [Schneider, L. S. Overview of generalized anxiety disorder in the elderly. J Clin. Psychiatry 57, 34-45, 1996]. Accordingly, such bi-directional interaction is important in the fact that anxiety and the loss of memory have a close relation with each other [Sinoff, G., Werner, P. Anxiety dosorder and accompanying subjective memory loss in the elderly as a predictor of future cognitive decline. Int. J. Geriatr. Psychiatry 18, 951-959, 2003]. There are comorbidities of anxiety and depression in 25-50% of patients, and such comorbidities make anxiety or depression more serious [Kessler R C et al., (1999) lifetime comorbidities between social phobia and mood disorders in the US national comorbidity survey. Psychol. Med 29, 555-567].

Currently, benzodiazepines reacting on a GABA receptor are used as an anti-anxiety agent, however, these are commonly accompanied by adverse side effects, such as anterograde amnesia, movement disorder, mental disorder, delirium and others. Acetylcholinesterase inhibitors are currently used as a therapeutic agent for dementia, however, acetylcholinesterase inhibitor therapy in dementia is not much effective. Tacrine broadly used causes apparent adverse side effects, such as abdominal cramps, nausea, vomiting, diarrhea and others, in ⅓ of tacrine-administered patients. Acetylcholinesterase inhibitors include donepezil, rivastigmine, galatamine and others. Since such donepezil, rivastigmine and galatamine also have adverse side effects, such as nausea, vomiting, diarrhea, insomnia and others, these are limited in their use [Harman J. G., Limbird, L. E. Goodman & Gilman's The Pharmacological basis of Therapeutics. 10th edition, McGraw Hill, 2001].

Tricyclic antidepressant, monoamine oxidase inhibitor (MAOI) and selective serotonin reuptake inhibitor (SSRI) and others are used as an antidepressant. Tricyclic antidepressant has harmful actions including insomnia, anxiety, fatigue, weakness, xerostomia, dilation of pupil and others, and even usual dose thereof can cause sudden cardiac death in patients with heart diseases due to ventricular arrhythmia or coronary thrombosis. MAOI causes hepatotoxicity and postural hypotension as an adverse side effect, and induces insomnia, agitation, clamps and others when administered overdose. SSRI induces gastrointestinal disorders such as diarrhea, nausea and vomiting, worry, anxiety, sleep disorder, weight, sexual dysfunction and withdrawal symptom, as an apparent adverse side effect [Harman J. G., Limbird L. E. Goodman & Gilman's The Pharmacological Basis of therapeutics. 10th edition, McGraw Hill, 2001]. Accordingly, it has been required to develop a new drug that has excellent therapeutic efficacy against anxiety and depression as well as memory impairment at the same time but with less adverse side effects.

Rubi Fructus is called raspberry or wild raspberry, and is the unripe fruit of Rubus coreanus MIQ., Rubus tokkura SIEBOID, Rubus crataegiofolius BUNGE, Rubus itoensis LEV. et VAN., Rubus parviflious L. var. triphyllus NAKAI, and Rubus chingii HU. Rubi Fructus is known to contains organic acids including malic acid, citric acid and tartaric acid; sugars including fructose and glucose; vitamins including vitamin C and vitamin-A like compounds; and triterpenoides including nigaichigoside F1, F2 (1,2), suavissimoside R1 (3), coreanoside F1 (4) and coreanogenic acid (5). Further, fupenzic acid, rubusoside, sanguiin H6, goshonoside F1, F2, F3, F4, F5, F6 and F7 are present therein. Also, it is known to have pharmacological actions including anti-bacterial action, astringent action and liver- and kidney-protecting action, and is thus used in the field of Chinese medicine to restore vigor and energy and treat gonacratia, frequency, weakness and fatigue. (Jeong B. S., Shin M. K. Dohaehyangyakdaesajeon, Younglimsa, 1998; Traditional Oriental Medicine Database (TradMed) Natural Products Research Institute (NPRI) of Seoul National University, 1999; Zhu, Y. P.Chinese Materia Medica. Chemistry, Pharmacology and Application, Harwood Academic Publishers, 1998).

However, it has been not reported that Rubi Fructus has prophylactic and therapeutic effects on anxiety, depression and dementia as well as memory-improving effect.

### BRIEF SUMMARY

The present inventor has studied about materials which can induce prophylactic and therapeutic effects on anxiety, depression and dementia as well as memory-improving effect in the moderns afflicted with brain damage caused by external environmental factors including various kinds of stresses, drinking alcohols, smoking cigarettes and others, and finally found that an extract of Rubi Fructus has excellent prophylactic and therapeutic effects on anxiety, depression and dementia, as well as memory-improving effect.

Accordingly, an object of the present invention is to provide a pharmaceutical composition and a dietary supplement, which have inhibitory effect on anxiety and depression, memory-improving effect and therapeutic effect on dementia, by using an extract of Rubi Fructus.

Another object of the present invention is to prevent and treat anxiety, depression and memory impairment interacting with each other, effectively by using an extract of Rubi Fructus that induces inhibitory effect on anxiety and depression as well as memory-improving effect.

Additional advantages, objects and features of the invention will be set forth in part in the description which follows and in part will become apparent to those having ordinary skill in the art upon examination of the following or may be learned from practice of the invention.

In order to accomplish these objects, there is provided a composition containing an extract of Rubi Fructus for preventing and treating anxiety, depression and dementia, and improving memory.

The composition for inhibiting anxiety and depression and improving memory contains an extract of Rubi Fructus 0.5-50 wt. % based on the total weight of the composition.

The extract of Rubi Fructus may be prepared by the following preparation process.

Step 1: Rubi Fructus is extracted with an organic solvent selected from the group consisting of lower alcohol having a carbon number of 1 to 4, lower acetate, such as ethylacetate, acetone, chloroform, dichloromethane, carbon tetra chloride, methylenechloride, ether or hexane, or a mixed solvent thereof, preferably a mixture of methane or methanol and water within a ratio of 1:0.2-1.5, at 5-80 □, preferably 30-55 □, for 15 minutes to 48 hours, preferably 30 minutes to 12 hours, in order to obtain a lower alcohol-soluble fraction.

Further, the extract of Rubi Fructus of the present invention may be additionally subjected to the following fractioning process that is involved in the conventional fractionation and separation method [Carborne J. B. Photochemical methods: A guide to modern techniques of plant analysis. 3rd Ed. pp 6-7, 1998].

Step 2: The lower alcohol-soluble fraction obtained in the above step 1 is dissolved in a mixed solvent of lower alcohol and water, followed by adjusting the pH to 2-4 and extracting with the same amount of chloroform, in order to obtain a chloroform-soluble fraction.

Step 3: The chloroform-insoluble fraction is adjusted to pH 9-12 with ammonium hydroxide, followed by extracting and fractioning with the same amount of a mixed solvent of chloroform/methanol, in order to obtain a chloroform/methanol-soluble fraction. Herein, a mixing ratio of chloroform:methanol is preferably 1:0.1-1. Alkaloids are present mostly in the chloroform/methanol-soluble fraction among the chloroform-insoluble fractions, while quaternary alkaloids and N-oxides are present in the methanol-soluble fraction among the chloroform/methanol-insoluble fractions.

Step 4: The chloroform/methanol-insoluble fraction is further extracted and fractioned with methanol in order to obtain a methanol-soluble fraction.

A therapeutic agent for inhibiting anxiety, depression and dementia, and improving memory of the present invention contains the lower alcohol-soluble fraction, the chloroform-soluble fraction, chloroform/methanol-soluble fraction and the methanol-soluble fraction.

The composition containing the extract of Rubi Fructus of the present invention may further contain appropriate carrier, excipient and diluent which include lactose, dextrose, sucrose, sorbitol, mannitol, xylitol, erythritol, maltitol, starch, acacia gum, alginate, gelatin, calcium, phosphate, calcium silicate, cellulose, methyl cellulose, microcrystalline cellulose, polyvinyl pyrrolidone, water, methylhydroxybenzoate, propylhydroxybenzoate, talc, magnesium stearate and minerals.

The composition containing the extract of Rubi Fructus may be formulated into oral formulations including power, tablet, capsule, suspension, emulsion, syrup and aerosol; topical formulations; suppositories; and sterile solution for injection, according to the conventional formulation methods.

The usual dose of the extract of Rubi Fructus may be altered according to age, gender and body weight of a patient, however, recommended dosage thereof is 0.1-500 mg/kg to be administered to a patient either once or several times daily. The dosage of the extract or fraction of Rubi Fructus can be either increased or decreased according to route of administration, disease states, gender, body weight, age and others. Accordingly, the present invention is not limited thereto.

The composition containing the extract of Rubi Fructus may be variously used for remedies, food, beverages and others. For instance, the extract of Rubi Fructus can be applied to various kinds of food, beverages, chewing gums, multivitamins, dietary supplements and others.

Since the extract of Rubi Fructus of the present invention has scarcely any toxicity and adverse side effect, it is capable of long-term administration for prophylactic purpose.

The extract of Rubi Fructus may be added to food or beverages for inhibiting anxiety and depression and improving memory. In this time, generally, a food composition of the present invention may contain 0.1-15 wt. %, preferably 1-10 wt. % of the extract of Rubi Fructus, based on the total weight thereof, while a beverage composition may contain 1 to 30 g, preferably 3 to 10 g per 100 ml.

The beverage composition contains the extract of Rubi Fructus as an essential ingredient according to the above ratios, but may further contain optionally flavors or natural carbohydrates such like the conventional beverages.

The natural carbohydrates include conventional saccharides including monosaccharides, such as glucose, fructose and the like; disaccharides, such as maltose, sucrose and the like; and polysaccharides, such as dextrin, cyclodextrin and the like, and sugar alcohol including xylitol, sorbitol, erythritol and the like. The flavors are advantageously used and include natural flavors including thaumatin and stevia extracts, such as revaudioside A, glycyrrhizin and the like; and synthetic flavors including saccharin and aspartame. The proportion of the natural carbohydrates ranges from 1 to 20 g, preferably 5-12 g per 100 ml composition of the present invention.

The composition of the present invention further may contain a variety of nutrients, vitamins, anti-oxidants, minerals (electrolytes), flavors including natural and synthetic flavors, colorants or filling agents (cheese, chocolate and others), pectic acid or its slats, alginic acid or its salts, organic acid, thickening agents (protective colloid agents), pH regulators, stabilizers, preservatives, glycerin, alcohols and carbonating agents used in carbohydrate beverages. Additionally, the composition of the present invention may contain fruit fleshes in order to manufacture natural fruit juices, fruit beverages and vegetable beverages. Such ingredients can be used individually or by being mixed together. Such additives are generally added to the composition in the ratio of from 0 to approximately 20 parts by weight per 100 parts by weight of the composition.

### BRIEF DESCRIPTION OF THE DRAWINGS

The above and other objects, features and advantages of the present invention will be more apparent from the following detailed description taken in conjunction with the accompanying drawing, in which:

FIG. 1 shows anti-anxiety effect of an extract of Rubi Fructus (Staircase test). Values, shown therein, are mean±standard deviation (n=10), and significance in the control group is defined as * *: P<0.01;

FIGS. 2a, 2b, 2c, and 2d show anti-anxiety effect of an extract of Rubi Fructus (Elevated plus maze test). Values, shown therein, are mean±standard deviation (n=10), and significance in the control group is defined as * :P<0.05; * *: P<0.01;

FIG. 3 shows inhibitory effect of an extract of Rubi Fructus on strychnine-induced seizure. Values, shown therein, are mean±standard deviation (n=7), and significance in the control group is defined as * :P<0.05; * *: P<0.01;

FIGS. 4a, 4b and 4c show an effect of an extract of Rubi Fructus on picrotoxin- and isoniazid-induced seizure. Values, shown therein, are mean±standard deviation (n=10);

FIGS. 5a and 5b show inhibitory effect of an extract of Rubi Fructus on the reuptake of monoamine;

FIGS. 6a and 6b show memory-improving effect of an extract of Rubi Fructus (Passive avoidance test). Values, shown therein, are mean±standard deviation (n=8-10), and significance in the control group is defined as * :P<0.05; and

FIG. 7 shows effect of an extract of Rubi Fructus on muscle relaxation (Rotarod test). Values, shown therein, are mean±standard deviation (n=10).

### DETAILED DESCRIPTION OF EXEMPLARY EMBODIMENTS

Hereinafter, preferred embodiments of the present invention will be described in detail with reference to the accompanying drawing. The aspects and features of the present invention and methods for achieving the aspects and features will be apparent by referring to the embodiments to be described in detail with reference to the accompanying drawings. However, the present invention is not limited to the embodiments disclosed hereinafter, but can be implemented in diverse forms. The matters defined in the description, such as the detailed construction and elements, are nothing but specific details provided to assist those of ordinary skill in the art in a comprehensive understanding of the invention, and the present invention is only defined within the scope of the appended claims. In the entire description of the present invention, the same drawing reference numerals are used for the same elements across various figures.

### Embodiment 1

### Preparation of the Extract of Rubi Fructus

Rubi Fructus 250 g was finely cut and then extracted with 70% methanol 750 ml using an ultrasound extractor, 3 times. The obtained extract was filtered and concentrated under reduced pressure using a rotary evaporator (EYELA N-N Series. The concentrate extract was freeze-dried in order to obtain a methanol co-extract 17.4 g.

### Experimental Example 1

### Anti-Anxiety Test (Staircase Test)

1) Procedure

The following anti-anxiety test was carried out according to a previous method (Simiand et al.) [Simiand, J., Jeane. P. E., Morre, M. The staircase test in mice: A simple and efficient procedure for primary screening of anxiolytic agents. Psychopharmacolgy 84, 48-53, 1984]. A mouse was placed on the floor of a staircase box (10 cm×45 cm×25 cm) with its tail to the staircase. The number of rears is counted over a 3 minute-period as an indicator of anxiety. A step is considered to be climbed only if the mouse had placed all four paws on the step. The number of steps descended is not taken into account, in order to simplify the observation. After each mouse had been tested, the staircase box was cleaned up not to stimulate the olfactory sense of the next mouse. The Rubi Fructus fraction was orally (P. O.) administered at a dose of 100 mg/kg within 60 minutes before starting the test, and all tests were carried out between 8 a.m. and 11 p.m.

2) Results

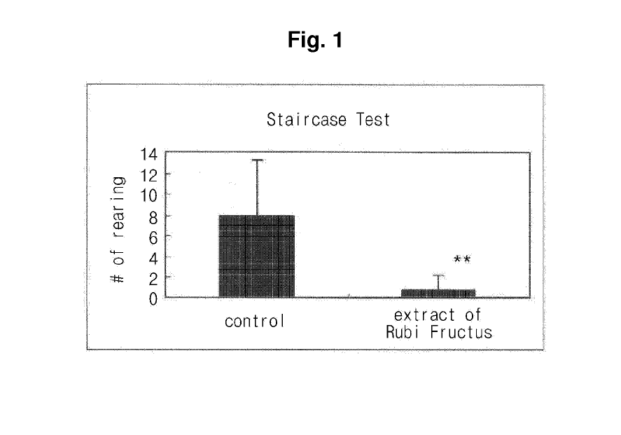

The result was illustrated in FIG. 1. The numbers of rearing as an indicator of anxiety were 0.7 times in the Rubi Fructus fraction-administered group and 8 times in the control group, respectively. Theses showed that the Rubi Fructus fraction remarkably decreased the number of rearing as compared to that of the control group. Accordingly, it is suggested that the Rubi Fructus fraction has remarkable anti-anxiety action.

### Experimental Example 2

### Anti-Anxiety Test (Elevated Plus Maze Test)

1) Procedure

Male ICR mice (20 g) were P.O. administered with the Rubi Fructus fraction 100 mg/kg, and then after 1 hour, subjected to elevated plus maze test. The elevated plus maze was 70 cm high and consisted of two open arms (50×10 cm) and two closed arms (50×10×40 cm) which crossed over each other [Pellow, S., Chopin, P. H., File, S. E, Briley, M. Validation of open:closed entries in an elevated plus maze as a measure of anxiety in the rat. J. Neurosci. Meth. 14, 149-167, 1985]. Mice were P. O. administered with the Rubi Fructus fraction 100 mg/kg, and after 1 hour, the mice were then placed in the center of the elevated plus maze to go toward the closed arms. The numbers of entries into the open and closed arms were measured over 5 minutes.

2) Results

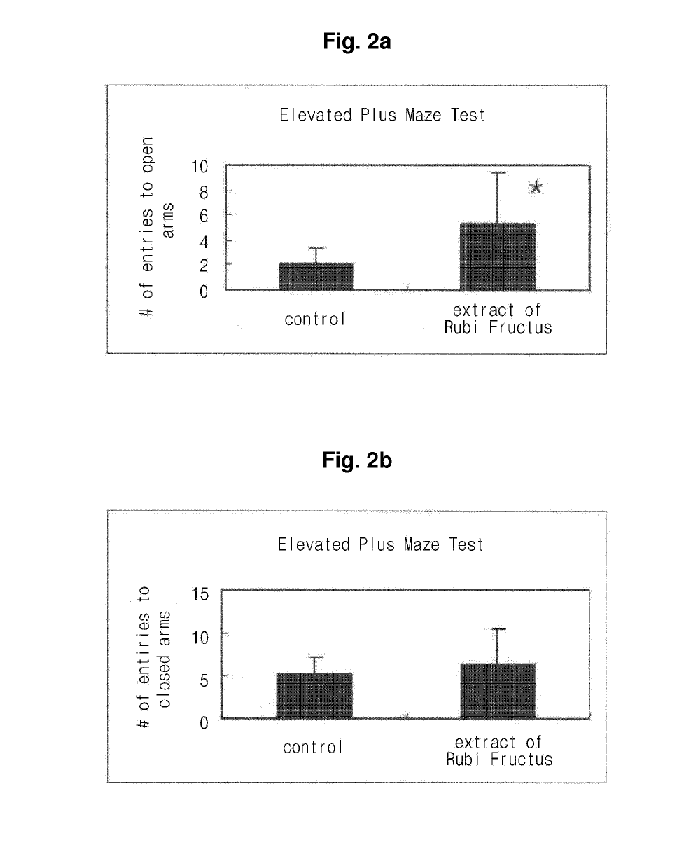

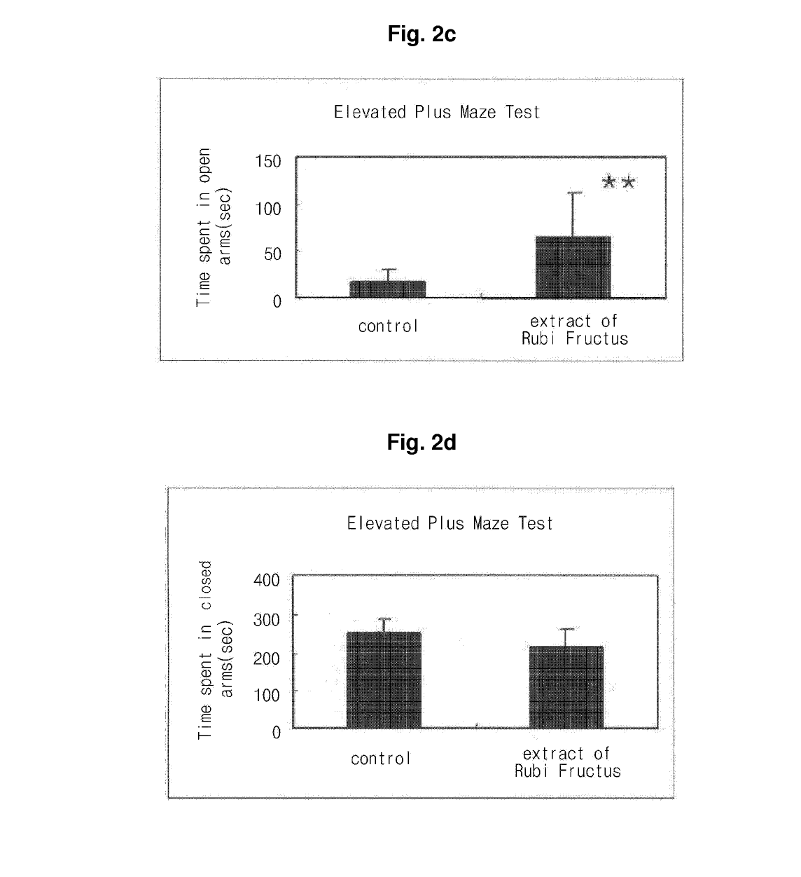

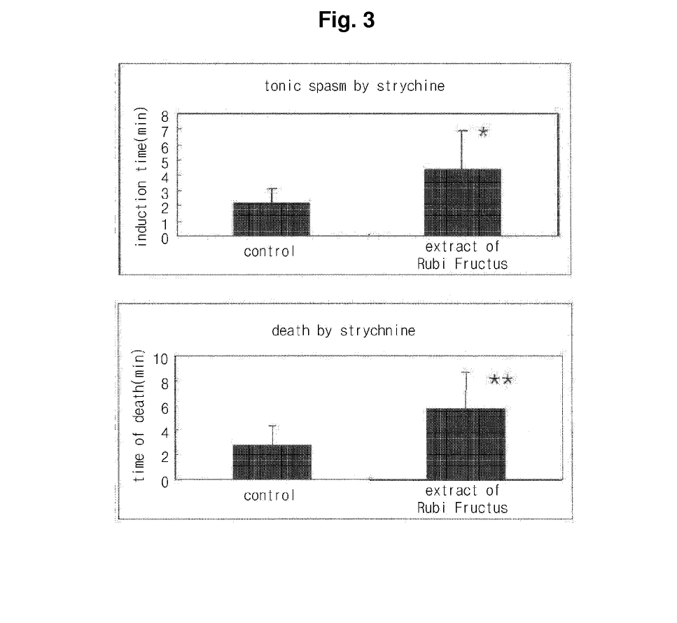

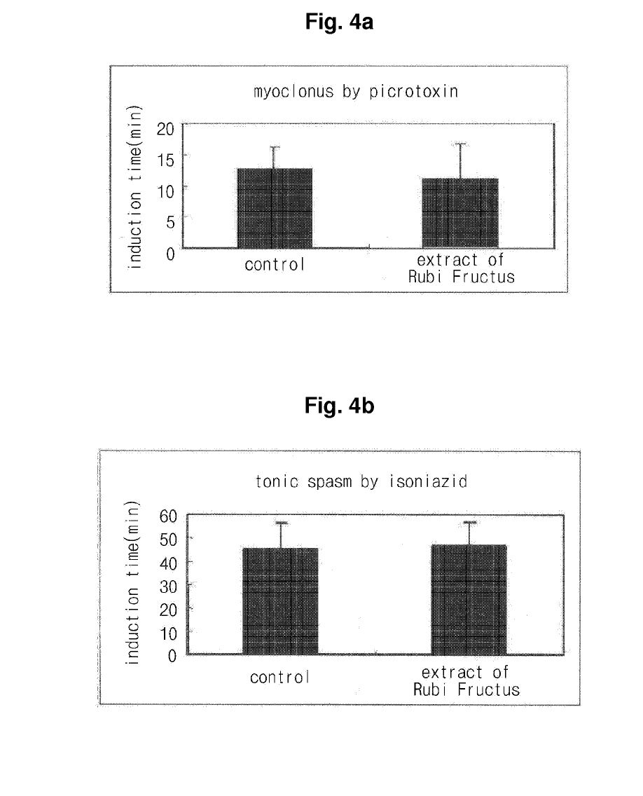

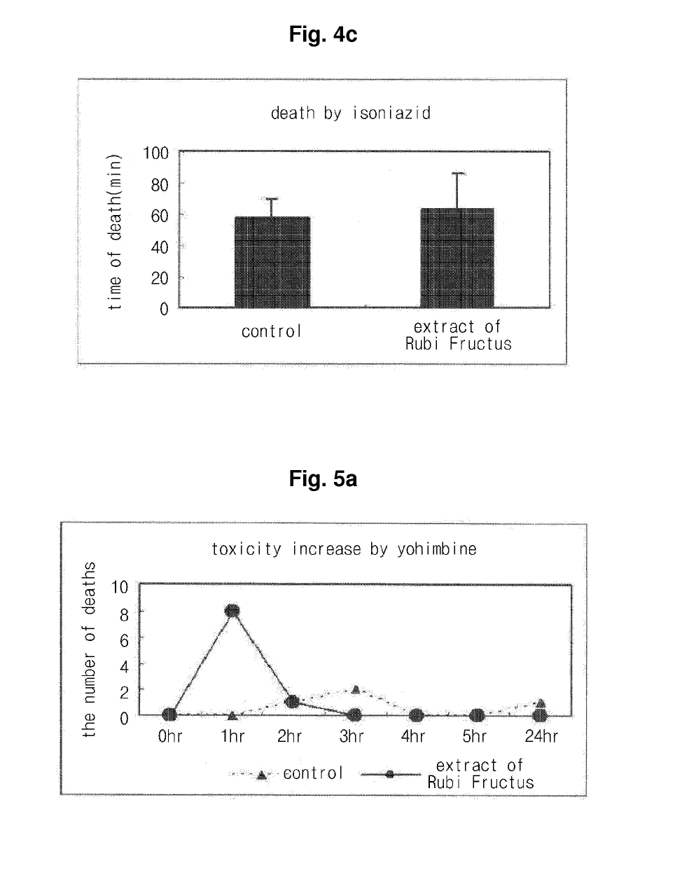

The Rubi Fructus fraction-administered group increased the number of entries into the open arms 2.65 times, as compared to that of the control group (FIG. 2a), however, there was no statistical significance of difference in the numbers of entries into the closed arms between the Rubi Fructus-administered group and the control group (FIG. 2b). The Rubi Fructus fraction-administered group increased the amount of time spent in the open arms 3.78 times, as compared to that of the control group (FIG. 2c), however, there was no statistical significance of difference in the amounts of time spent in the closed arms between the Rubi Fructus-administered group and the control group (FIG. 2d). In the elevated plus maze test, anti-anxiety effect increases the number of entries into the open arms and the time spent in the open arms, and the number of entries into the closed arms and the time spent in the closed arms are considered as indicators of exploratory activity. Accordingly, it is suggested that the extract of Rubi Fructus induces remarkable anti-anxiety effect.

### Experimental Example 3

### Examination for the Mechanism of Antidepressant Action of Rubi Fructus (Enhancement of Yohimibine Induced Toxicity)

1) Procedure

Male ICR mice (20 g) were P.O. administered with the extract of Rubi Fructus (100 mg/kg), and 30 minutes later, yohimbine (25 mg/kg) was then subcutaneously injected thereinto. Then after 1 hour, 2 hours, 3 hours, 4 hours, 5 hours and 24 hours, the death rate was estimated [Goldberg M R, Robertson D (1988) Influence of alpha stimulants and beta blockers on yohimbine toxicity. Prog neuro-Psychopharmacol Biol Psychiat 12, 569-574].

It has been reported that yohimbine acts as an antagonist of an α2-receptor, and stimulates the release of monoamines (noerepinephrine, serotonin, dopamine). Further, it acts as an agonist of a serotonin receptor [Feuerstein T J, Hertting G, Jackisch R (1985) Endogenous noradrenaline as modulator of hippocampla serotonin (5-HT)-release. Dual effects of yohimbine, rauwolscine and corynanthine as alpha-adrenoceptor antagonists and 5-HT-receptor agonist. Naunyn schmiedebergs Arch Pharmacol 329, 216-221]. The tricyclic antidepressant blocks the reuptake of norepinephrine, serotonin and dopamine, and therefore, inhibits physiological inactivation thereof at the axon terminal and shows antidepressant action. It was revealed that the concurrent administration of imipramine inducing antidepressant action by inhibiting the reuptake of serotonin and yohimbine increased the concentration of serotonin at the axon terminal, and laboratory animals would die from the toxicity thereof. Therefore, the mechanism of action of yohimbine is used for exploring antidepressants [Quinton R M (1963) The increase in toxicity of yohimbine induced by imipramine and other drugs in mice. Br J Pharmacol 21, 51-66].

2) Results

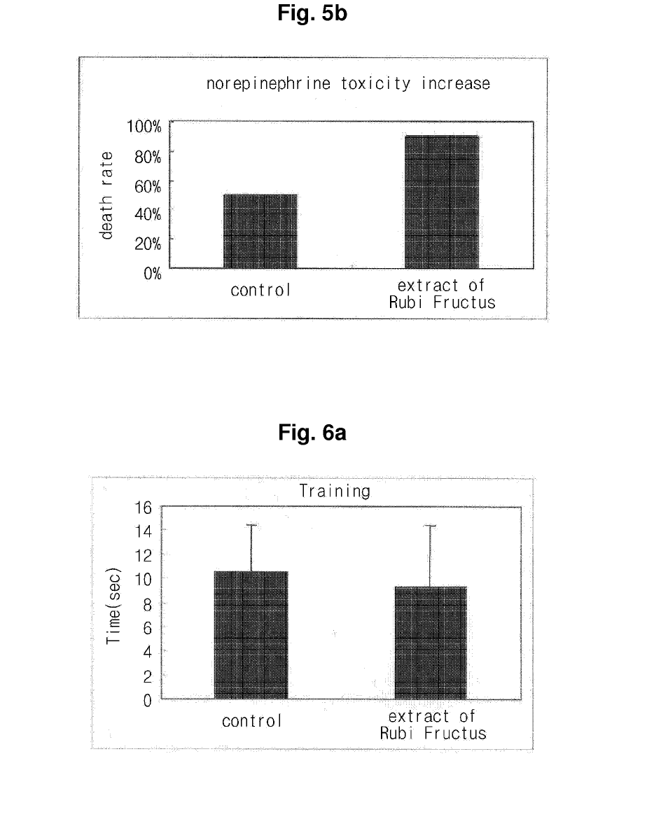

In the control group administered with only yohimbine, the death rate was 10%, 2 hours after administration, however, in the experimental group administered with the extract of Rubi Fructus then yohimbine sequentially, the death rate was 90%, 2 hours after administration (FIG. 5a). 24 hours after administration of yohimbine, the total death rates were, respectively, 40% in the control group and 90% in the experimental. This showed that the extract of Rubi Fructus blocked the reuptake of monoamine at the axon terminal, and therefore, enhanced the actions of yohimbine, as an antagonist of α2-receptor and an agonist of a serotoin receptor, and remarkably increased the death rate. Accordingly, it is considered that the extract of Rubi Fructus blocks the reuptake of norepinephrine, serotonin and dopamine, and therefore, enhanced the action of such monoamines and the action of serotonin as an agonist to induce antidepressant effect.

### Experimental Example 4

### Examination for the Mechanism of Anti-Anxiety Action of Rubi Fructus (Potentiation of Norepinephrine Toxicity)

1) Procedure

Male ICR mice (20 g) were P.O. administered with the extract of Rubi Fructus (100 mg/kg), and 1 hour later, norepinephrine (3 mg/kg) was then subcutaneously injected thereinto. The mice were placed into a plastic cage and fed food and water freely. Then after 48 hours, the death rate was estimated (Alperman H G, Schacht U, Usinger P, Hock F J (1992) Drug Dev Res 25, 267-282, 1 Psychiat 12, 569-574). The antidepressant blocks the reuptake of norepinephrine and other physiological amines, and therefore, inhibits physiological inactivation thereof at the axon terminal and shows antidepressant action. Accordingly, the concurrent administration of a medicinal substance inducing antidepressant action by inhibiting the reuptake of norepinephrine and norepinephrine increases the concentration of norepinephrine at the axon terminal, and laboratory animals will die from the toxicity thereof.

2) Results

The death rates were, respectively, 50% in the control group and 90% in the experimental group that was administered with the extract of Rubi Fructus and norepinephrine sequentially (FIG. 5b). This showed that the extract of Rubi Fructus blocked the reuptake of norepinephrine at the axon terminal, and therefore, enhanced the actions of norepinephrine remarkably. Accordingly, it is considered that the extract of Rubi Fructus induces the mechanism blocking the reuptake of norepinephrine at the axon terminal and also thus induces antidepressant action.

### Experimental Example 5

### Passive Avoidance Test: Memory-Improving Test

1) Procedure

Male ICR mice (20 g) were P.O. administered with the Rubi Fructus fraction 100 mg/kg, and then after 1 hour, subjected to passive avoidance test using Gemini avoidance system (San Diego Instruments, USA). The following test was carried out according to a slight modification of a previous method (Kumar et al.) [Kumar, V., Singh, P. N., Muruganandan, A. V., Bhattacharya. Effect of Indian Hypericum perfortatum Linn on animal models of cognitive dysfunction. J Ethnopharmacology 72, p 119-128, 2000].

On the first day of training test, mice were placed into a lightened room. Then, after they were acclimated thereto for 300 seconds, they were allowed to move to a dark box through a guillotine door and received a punishing electrical shock (0.3 mA) for 1 second. After 24 hours, in the experimental test, the mice were acclimated to the lightened box for 30 seconds and then allowed to move to the dark box. At this time, the latency times for moving to the dark box were measured. A punishing electrical shock was excluded from the test on the second day. If the mice did not move to the dark box within 180 seconds, a maximum point of 180 is allowed [Mohamed, A. F., Matsumoto, K., Tabata, K., Takayama, H., Kitajima, M., Aimi, N., Watanabe, H. Effects of Uncaria tomentosa total alkaloid and its components on experimental amnesia in mice: Elucidation using the passive avoidance test. J. Pharm. Pharmacol. 52, 1553-1561, 2000].

2) Results

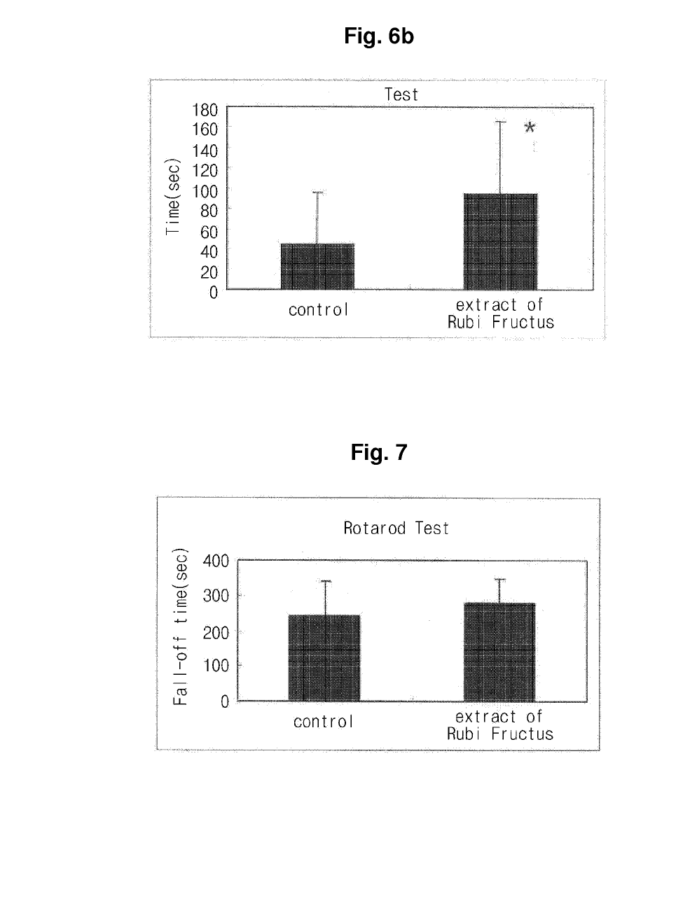

According to the result of the training test on the first day, as shown in FIG. 6a, there was no statistical significance of difference between the groups. However, according to the result of the experimental test on the second day, as shown in FIG. 6b, the extract of Rubi Fructus-administered mice remarkably increased memory-improving effect 2.08 times, as compared to that of the control group.

### Experimental Example 6

### Muscle Relaxation Test (Rotarod Test)

1) Procedure

Male ICR mice (20 g) were P.O. administered with the extract of Rubi Fructus (100 mg/kg). After 1 hour, the mice were placed on Rotarod (diameter 3 cm, rotating spindle speed 15 rpm) and the latency times for falling down were measured.

2) Results

There was no statistical significance of difference between the extract of Rubi Fructus-administered group and the control group (FIG. 7). It is suggested that the extract of Rubi Fructus has no muscle relaxation effect. Further, it is proved that the muscle relaxation effect is not related to a decrease of the number of rearing in the staircase test; decreases of the number of entries into the open arms and times spent therein in elevated plus maze tests; and an increase of time for moving to the dark box after 24 hours in the passive avoidance test.

### Experimental Example 7

### Oral Toxicity Test of Rubi Fructus Extract

1) Procedure

30 male ICR mice (approximate 20 g) were fed at a temperature of 23□, a relative humidity of 50% and an illumination of 150-300 Lux, in the animal room for a week, then divided into 4 groups consisted of 4.

The methanol extract of Rubi Fructus was P. O. administered to 10 mice per group at doses of 0 mg/kg, 50 mg/kg, 500 mg/kg and 5,000 mg/kg, respectively. After administration, the mice were observed over 7 days to check changes in general condition and the number of deaths. On 7th day of administration, the mice were subjected to euthanasia and an autopsy in order to examine internal organs thereof with the naked eye.

2) Results

There were no abnormal findings due to administration of the extract of Rubi Fructus. Moreover, there were no dead mice even when administered with the extract of Rubi Fructus, at a dose of 5,000 mg/kg. No signs of toxicity were detected in each organ during tissue biopsy, and the organs appeared to be safe from the extract of Rubi Fructus.

Hereinafter, example formulations of the above described pharmaceutical composition will be described, however the present invention is not limited thereto.

### Formulation Example 1

### Tablet

The below ingredients were formulated into a tablet by the conventional manufacturing method of tablet.

| | | |
|---|---|---|
| Methanol extract of Rubi Fructus | 500.0 | mg |
| Lactose | 500.0 | mg |
| Talc | 5.0 | mg |
| Magnesium stearate | 1.0 | mg |

### Formulation Example 2

### Capsule

The below ingredients were formulated into a capsule by the following process.

The extract of Rubi Fructus was mixed with pharmaceutical excipient, and the mixture was then filled into a gelatin capsule

| | | |
|---|---|---|
| Methanol extract of Rubi Fructus | 500.0 | mg |
| Starch 1500 | 10.0 | mg |
| Magnesium stearate BP | 100.0 | mg |

### Formulation Example 3

### Syrup

The below ingredients were formulated into syrup by the following process.

Refined sugar was dissolved in purified water, first. P-oxybenzoate, p-oxypropylbenzoate and the extract of Rubi Fructus were added to the refined sugar-dissolved solution, then followed by dissolving at 60 □ and cooling. Finally purified water was added to the above solution to make 150 ml of a solution.

| | | |
|---|---|---|
| Methanol extract of Rubi Fructus | 5.0 | mg |
| Refined sugar | 95.1 | g |
| P-oxybenzoate | 80.0 | mg |
| P-oxypropylbenzoate | 16.0 | mg |
| Total amount of purified water added | 150 | ml |

### Formulation Example 4

### Solution

The below ingredients were formulated into solution by the conventional manufacturing method of solution, and then filled into a brown bottle to obtain solution.

| | | |
|---|---|---|
| Methanol extract of Rubi Fructus | 500.0 | mg |
| Isomerized glucose syrup | 20.0 | g |
| Antioxidant | 5.0 | mg |
| Methyl p-oxybenzoate | 2.0 | mg |
| Total amount of purified water added | 100.0 | ml |

### Formulation Example 5

### Powder

The below ingredients were mixed by the conventional manufacturing method of powder, and then inserted into a medicine envelope to obtain powder.

| | | |
|---|---|---|
| Methanol extract of Rubi Fructus | 50.0 | mg |
| Lactose | 100.0 | mg |
| Talc | 5.0 | mg |

### Formulation Example 6

### Solution for Injection

The below ingredients were filled into a 2.0 ml ampoule by the conventional manufacturing method of solution for injection to obtain sterile solution for injection.

| | | |
|---|---|---|
| Methanol extract of Rubi Fructus | 50.0 | mg |
| Antioxidant | 1.0 | mg |
| Twin 80 | 1.0 | mg |
| Total amount of purified water added | 2.0 | ml |

Further, a dietary supplement was manufactured by following method.

[Manufacture of Whole Grain Food]

The grains of unpolished rice, barley, glutinous rice and job's tear were pregelatinized, and then dried. The dried grains were pulverized into a particle size of 60 mesh after electric power distribution to obtain powder. Further, black beans, black sesame seeds and perilla seeds were steamed by the well-known method, and then dried. The dried seeds were pulverized into a particle size 60 mesh after electric power distribution to obtain powder.

The above manufactured powders of grains and seeds were mixed with the dried powdered extract of Rubi Fructus, according to the following ratio.
[Grains: unpolished rice 30 wt. %, job's tear 15 wt. %, barley 20 wt. %, Seeds: perilla seeds 7 wt. %, black beans 8 wt. %, black sesame seeds 7 wt. %, The dried powdered extract of Rubi Fructus: 3 wt. %, Ganoderma lucidum 0.5 wt. %, Chinese foxglove 0.5 wt. %]

As described above, the composition containing an extract of Rubi Fructus for preventing and treating anxiety, depression and dementia and improving memory, according to the present invention, produces the following effects.

First, the composition containing the extract of Rubi Fructus induces prophylactic and therapeutic effects on anxiety, depression and dementia, and shows memory-improving effect

Second, the composition containing the extract of Rubi Fructus is useful for people afflicted with anxiety, depression and declined memory among the moderns who have brain damage risk by various kinds of environmental stresses.

It should be understood by those of ordinary skill in the art that various replacements, modifications and changes in the form and details may be made therein without departing from the spirit and scope of the present invention as defined by the following claims. Therefore, it is to be appreciated that the above described embodiment are for purposes of illustration only and are not to be construed as limitations of the invention.

1-10. (canceled)

11. A method of treating anxiety, depression, dementia, and memory decline in a mammal in need thereof, the method comprising:
administering to the mammal an extract of Rubi Fructus.

12. The method of claim 11, wherein the extract of Rubi Fructus is administered to the mammal in need thereof in a dosage of 0.1 mg to 500 mg of extract per kilogram of the mammal.

13. The method of claim 12, wherein the extract of Rubi Fructus is administered to the mammal in need thereof once daily.

14. The method of claim 12, wherein the extract of Rubi Fructus is administered to the mammal in need thereof more than once a day.

15. The method of claim 11, wherein the extract of Rubi Fructus is obtained by extracting Rubi Fructus with an organic solvent selected from the group consisting of lower alcohol having a carbon number of 1 to 4, alcohol-water mixture, lower acetate, such as ethylacetate, acetone, chloroform, dichloromethane, carbon tetra chloride, methylenechloride, ether or hexane, or a mixed solvent thereof.

16. The method of claim 11, wherein the extract of Rubi Fructus is obtained by extraction with a 70% methanol solvent in an ultrasound extractor.
